(19) United States
(12) Patent Application Publication
(10) Pub. No.: US 2022/0241443 A1
(43) Pub. Date: Aug. 4, 2022

(54) HANDLE ASSEMBLY

(71) Applicant: Purified Handle Co., Ypsilanti, MI (US)

(72) Inventors: John Krzeminski, FENTON, MI (US); Michael Ramsdell, Pinckney, MI (US); AJ Ward Hackert, Elkhart Lake, WI (US)

(21) Appl. No.: 17/591,896

(22) Filed: Feb. 3, 2022

Related U.S. Application Data
(60) Provisional application No. 63/145,131, filed on Feb. 3, 2021.

(51) Int. Cl. A61L 2/10; F21V 23/04

(52) U.S. Cl. CPC A61L 2/10; F21Y 2115/10; F21V 23/0464; F21V 23/0471

(57) ABSTRACT

A handle assembly includes a first support portion, a second support portion, and a rod portion extending between the first support portion and the second support portion. The rod portion having an outer surface. The handle assembly also includes a sensor array disposed in one of the first or second support portions. The sensor array is configured to detect if the handle assembly has been used. The handle assembly also includes a light board configured to produce germicidal UV light upon detection from the sensor array that the handle assembly has been used such that the handle assembly is configured to automatically sanitize the outer surface of the rod portion after the handle assembly has been used.

### RELATED APPLICATIONS

The present disclosure is related to and claims priority to U.S. Provisional No. 63/145,131 filed Feb. 3, 2021.

### TECHNICAL FIELD

The present disclosure generally relates to preventing the spread of pathogens. More specifically, the present disclosure describes a UV light self-sanitizing door handle.

### BACKGROUND

In most modern buildings, ingress or egress is achieved by opening or closing a door via a doorknob, handle, push bar or equivalent. The interface at which a human makes contact with the door hardware is typically made with bare hands. Bare hands or even other physical surfaces that may be used to contact the door handle have the potential to carry some sort of bacteria or pathogen. This in turn could be spread to the next user. The CDC estimates that the annual burden of the flu alone in the United States from 2017-2018 was: 9,300,000-45,000,000 illnesses, 140,000-810,000 hospitalizations, and 12,000-61,000 deaths. Since a variety of harmful pathogens may be transmitted through regular and direct contact with a shared surface like a door handle, the need for devices and mechanisms that mitigate the risk of contact exposure are necessary. Previously, a handle would have no ability to self-clean and would require a secondary sanitizing agent to be applied to the handle via a manual operation. This is unlikely to happen and requires direct input from an operator to complete. One attempt at solving this issue is disclosed in patent application no. US20080263820A1. It discloses a rotary drive unit that rotates the handle surface segments through a solution housed within the handle. However, the door handle may be wet during sequential uses and a disinfecting solution must be maintained within the handle making the solution impractical for high-traffic and high-volume applications. The psychological effects of a consistently moist door handle alone are enough to lead to a reduced use. The main point of a door and a door handle are to use them, not to think twice about having to use them. The presently disclosed handle has the potential for an opposite psychological impact, by becoming synonymous with a clean, bright and dry surface on which to grasp. All public or private buildings that have manual doors and want to limit the exposure of the user/operator to pathogens would use the disclosed handle. Key demographic would be businesses, government buildings, places of gathering, etc. that have high traffic and are without automated doors. Many buildings, whether historic, non-historic or new construction could benefit from the addition of the handle as disclosed herein. The main benefit is the reduced risk of contact with pathogens left on the surface of the handle by previous users or contact with other contaminated surfaces. The other benefits are, but not limited to, the inherent illumination of walkways, indication of state (of the handle), indication of state/openness (of the business or building), etc. It is an object of the handle assembly disclosed herein to safely sanitize and maintain an effectively clean surface for physical human contact. It is another object of the present handle to create a mechanism in which to better understand human interaction, study use cases, and visually display information to users. The disclosed handle reduces the risk of spreading pathogens on high traffic surfaces that would otherwise be infrequently cleaned or cleaned manually. By removing the human element from the equation, the handle as disclosed herein can maintain a sanitized surface that would reduce or limit the likelihood of viral and bacterial transmission from physical contact.

### SUMMARY

One aspect of the disclosure provides a handle assembly which includes a first support portion, a second support portion, and a rod portion extending between the first support portion and the second support portion. The rod portion having an outer surface. The handle assembly also includes a sensor array disposed in one of the first or second support portions. The sensor array is configured to detect if the handle assembly has been used. The handle assembly also includes a light board configured to produce germicidal UV light upon detection from the sensor array that the handle assembly has been used such that the handle assembly is configured to automatically sanitize the outer surface of the rod portion after the handle assembly has been used.

Another aspect of the disclosure provides the outer surface of the rod portion is comprised of quartz. A further aspect of the disclosure provides the outer surface of the rod portion is a rough surface.

Another aspect of the disclosure provides germicidal UV light is projected from the light board onto the rod portion in a direction perpendicular to the outer surface. A further aspect of the disclosure provides the germicidal UV light is projected using fiber optics to transmit light to the outer surface of the rod portion. Another aspect of the disclosure provides the germicidal UV light projected on to the outer surface of the rod portion comprises UV-C and visible light into the rod portion.

A further aspect of the disclosure provides the sensor array comprises a time of flight sensor or a radar sensor to sense distance.

Another aspect of the disclosure provides the first portion further comprises a light board including UV germicidal LEDs, light Spectrum LEDs, and a light sensor configured to sense ambient and UV light.

A further aspect of the disclosure provides the first portion further comprises an electronics carrier.

Another aspect of the disclosure provides the first portion further comprises an electronics cover configured to cover the electronics carrier.

A further aspect of the disclosure provides in the first portion further comprises an electronics board configured to control operation of the handle assembly.

Another aspect of the disclosure provides the electronics board further comprises control electronics, voltage regulators, and a motion sensor.

A further aspect of the disclosure provides the first portion further comprises a stanchion.

Another aspect of the disclosure provides the first portion further comprises a sensor cover comprised of a material capable of transmitting IR light and electronic signals for proximity sensing.

The details of one or more implementations of the disclosure are set forth in the accompanying drawings and the description below. Other aspects, advantages, purposes, and features will be apparent upon review of the following specification in conjunction with the drawings.

### BRIEF DESCRIPTION OF THE DRAWINGS

FIG. 1 is a perspective view of a handle assembly.

FIG. 2 is an exploded front perspective view of the handle assembly.

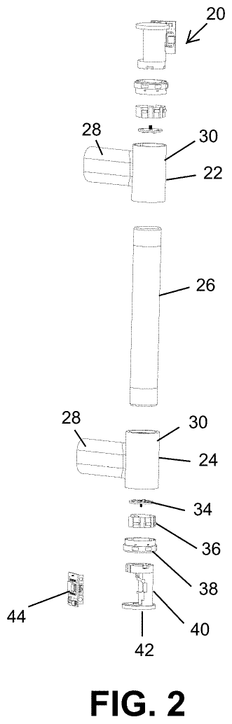

FIG. 3 is an exploded perspective view of a first portion of the handle assembly.

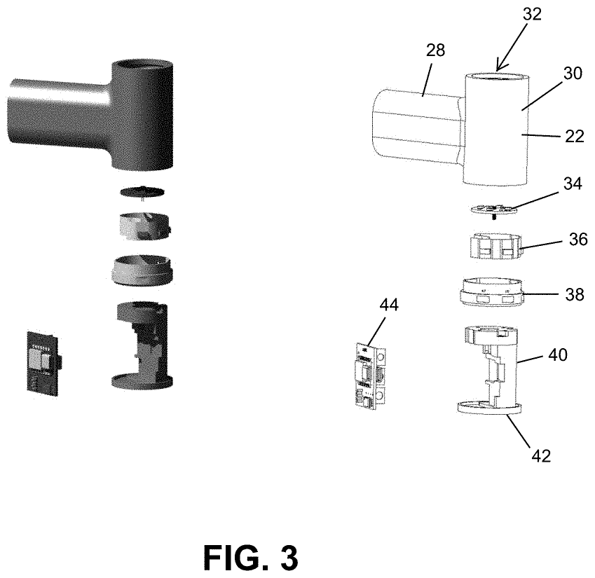

FIG. 4 is a partially exploded perspective view of the first portion of the handle assembly.

FIG. 5 is a schematic view of an outer surface of the rod portion of the handle assembly.

FIG. 6 is a schematic view of light transmission on the outer surface of the rod portion of the handle assembly.

Like reference numerals indicate like parts throughout the drawings.

### DETAILED DESCRIPTION

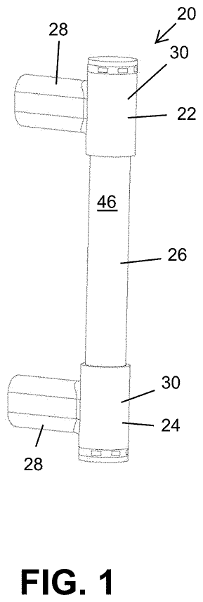

Referring now to the drawings and the illustrative examples depicted therein, a handle assembly 20 as described herein uses UV light from an internal source to produce a sanitized surface for physical contact. As used herein, the term UV light may refer to UV-A, UV-B, and/or UV-C light. Typically, germicidal UV light is UV-C light, however, some pathogens do react to UV-A and/or UV-B light. UV light is used to deactivate viruses, bacteria, fungi, spores, mold and other pathogens. Referring still to the example shown in FIG. 1, the handle assembly 20 may be coupled to another surface such as a door, a cabinet, or other movable surface. The handle assembly 20 is configured to be engaged by a user to open or close the movable surface such as the door. It is also contemplated that the principles described herein with respect to the handle assembly 20 may be used on other hard surfaces which may be subject to frequent contamination requiring sanitization.

Referring still to the example shown in FIG. 1, the handle assembly 20 includes a first support portion 22 and a second support portion 24 having a rod portion 26 extending between the first support portion 22 and the second support portion 24. In one example, the first support portion 22 and the second support portion 24 are identical, however, other configurations have been contemplated including the first support portion 22 being different from the second support portion 24. As such, mentions of the first support portion 22 herein may also be relevant to the second support portion 24, or may only be relevant to the second support portion 24. In one example, the first support portion 22 is a metallic or rigid body that houses various other handle assembly 20 components and is configured to suspend the rod portion 26, along with the second support portion 24. In some examples, the first support portion 22 and the second support portions 24 are mounted to a door or other surface as described above via a retaining stud or bolt. Various other coupling mechanisms have also been contemplated. In one example, one or more of the first support portion 22 or the second support portion 24 are coated in an anti-microbial coating.

Referring still to the example shown in FIG. 1 the first support portion 22 is a standard stanchion. In the example shown, the first support portion 22 includes a securing portion 28 configured to be secured to a surface. In some examples, the first support portion 22 and the second support portion 24 are mounted to a door or other surface as described above via a retaining stud or bolt. Various other coupling mechanisms have also been contemplated. The securing portion 28 extends perpendicularly from the coupling surface and is configured to allow the rod portion 26 to be spaced from the coupling surface. In one example, the first support portion 22 and the second support portion 24 have identical lengths, however, first support portions 22 and second support portions 24 having varying lengths have been contemplated. In some examples, the securing portion 28 is solid, however, it is also contemplated that the securing portion 28 may be hollow or partially hollow and configured to house wiring or other components.

The first support portion 22 also includes a rod coupling portion 30 which is generally cylindrical and having an aperture 32 disposed therethrough. The aperture 32 is configured to secure the rod portion 26 thereto. In one example, the rod portion 26 is only disposed partially within the aperture 32 such that the remaining portions of the aperture 32 are configured to house additional components of the handle assembly 20. While the securing portion 28 and the rod coupling portion 30 are both illustrated in FIG. 1 as being generally cylindrical having rounded edges, various other shapes and sizes have been contemplated including sharp edges, or a combination or rounded and sharp edges, as desired.

The rod coupling portion 30 also acts has a housing for various other components of the handle assembly 20 including but not limited to a light board 34, a sensor array 36, a sensor cover 38, an electronics carrier 40, an electronics cover 42, and a main electronics board 44. These components are shaped and sized to be placed within the aperture 32 of the rod coupling portion 30, as best illustrated in the figures. It is also contemplated that the rod coupling portion 30 may be a variety of shapes as described above, in which case, the components housed within the rod coupling portion 30 may be correspondingly shaped and sized. The light board 34 is the source of the light and contains UV germicidal LEDs along with light spectrum LEDs (white and RGB). The light board 34 also includes a light sensor for diagnostic purposes such as sensing ambient light and/or UV light.

The sensor array 36 includes a variety of sensors configured to sense time, distance, motion, and/or other measurables within a defined distance, called the safety parameter which is described in more detail below. In one example, the sensor array 36 is configured to detect human proximity to determine if it is safe to sanitize. In one example, the sensor array 36 includes a time of flight sensor and/or radar sensor to sense a user's distance from the device. Additionally, the sensor array 36 may be comprised of a flex PCB material in order to accommodate the shape of the sensors and of the aperture 32 of the rod coupling portion 30. It is also contemplated that the sensor array 36 may be composed of another material and/or a combination of materials. Additionally, the sensor cover 38 may be made from a material capable of transmitting IR light and radar signals for proximity sensing. The sensor array 36 and the sensor cover 38 are corresponding shaped such that the sensor cover 38 is placed over the sensor array 36 to protect the sensor array 36 and to secure the sensor array 36 in place. The main electronics board 44 is also configured to be disposed within the rod coupling portion 30. The main electronics board 44 contains control electronics and voltage regulators for the sensors and LEDs described above and is configured to control operation of the handle assembly 20. The main electronics board 44 may also include motion sensors used to detect whether the handle assembly 20 has been operated. In one example, the main electronics board 44 is a printed circuit board with hardware to drive the LEDs on the UV light source boards, a microcontroller, a BT module and supporting hardware, capacitive sensor for managing the proximity sensing of users, humidity and temperature sensors for monitoring environment, an accelerometer for door position, etc.

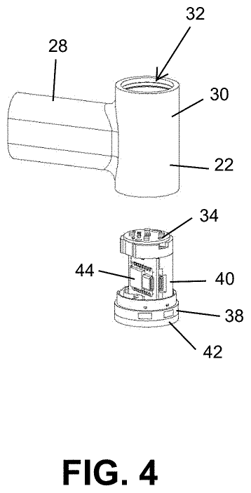

Referring still to the example shown in FIG. 4, the electronics carrier 40 and the electronics cover 42 are disposed within the rod coupling portion 30. More specifically, in the example shown in FIG. 4, the electronics carrier 40 is configured to have the light board 34 mount to a top surface and secures the LEDs contained in the light board 34 to project into the rod portion 26. Moreover, the electronics carrier 40 is configured to secure the main electronics board 44 and any voltage regulators. Moreover, the electronics carrier 40 is configured to hold the sensor array 36 and the sensor cover 38. The electronics carrier 40 may include protrusions or indents configured to help secure other components. In one example, the electronics carrier 40 is comprised of aluminum, however, various other materials have also been contemplated including but not limited to a plastic polymer or a metallic material. The electronics cover 42 is disposed on one end of the electronics carrier 40 and is configured to provide a barrier between the rod portion 26 and the remaining components disposed within the rod coupling portion 30.

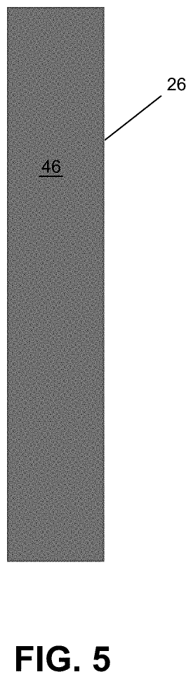

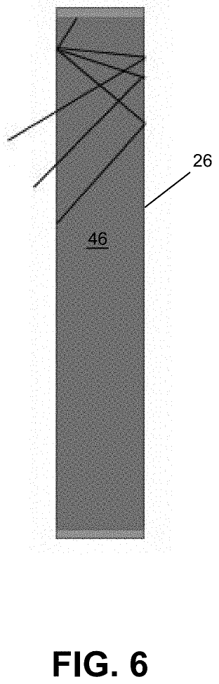

Referring still to the example shown in FIG. 1, the handle assembly 20 also includes the rod portion 26 extending between the first support portion 22 and the second support portion 24. The rod portion 26 has an outer surface 46 which is configured to be engaged by a user to open the door or other object which the handle assembly 20 is affixed. Moreover, once the surface is engaged by the user, the outer surface 46 of the rod portion 26 is automatically sanitized by germicidal UV light produced by the light board 34 and transmitted to the outer surface 46 of the rod portion 26. In one example, the rod portion 26 is generally cylindrical in shape, however, various other shapes have been contemplated including but not limited to a rectangular shape. The rod portion 26 is configured to be inserted into the first support portion 22 and the second support portion 24 and partially disposed in the aperture 32 of each. In other words, the rod portion 26 is suspended between the first support portion 22 and the second support portion 24 and is configured to be spaced from and parallel with the surface which the handle assembly 20 is affixed. Referring now to the example shown in FIGS. 5 and 6, germicidal UV light from the light board 34 is refracted to the outer surface 46 of the rod portion 26 by projecting light at an angle and making use of the material's Brewster angle (the angle of incidence at which light is perfectly transmitted through a material with no reflection). In one example, the outer surface 46 of the rod portion 26 is compressed of a frosted or fused quartz material, however, various other materials have been contemplated including fused silica or other UV transmitting materials. If the outer surface 46 was a clear quartz with no modifications, the rod portion 26 will act as a fiberoptic rod and transmit light through the rod portion with little to no loss, meaning no light is output onto the outer surface 46. Therefore, a diffusion methodology was needed to refract light onto the outer surface 46. Making the outer surface 46 rough, i.e. frosted, provides a method to refract light. Each imperfection forces a different projection angle and light emits onto the outer surface 46. Moreover, light is projected into the outer surface 46 of the rod portion 26 perpendicular to the handle surface. Further, light is projected to the outer surface 46 using fiberoptic principal to transmit both germicidal UV light and visible light into the handle and refract light onto the outer surface 46 of the rod portion 26.

UV light is transmitted through the rod portion 26 emitting energy over the outer surface 46. UV light is widely accepted to deactivate a multitude of bacteria, viruses, and other pathogens. The handle assembly 20 may be powered through a wired connection or unwired power source i.e., batteries, capacitor pack, solar panels, or other sources. In general operation, the handle assembly 20 uses onboard sensors to activate and deactivate the UV light and change the state of the RGBW light. The UV light is activated when a trigger event is detected (i.e., contact between matter and the handle is made, and/or the door position is changed from steady state). The UV light is deactivated when the proximity sensor or sensors detect matter within a set perimeter while the UV light is being produced. The RGBW light shows a predetermined light band during steady state and can be changed to show other states like: precleaning delay, cleaning, matter detected, etc. Other onboard hardware and sensors within the handle assembly 20 allow the handle to be connected to Wi-Fi and/or Bluetooth for data transmission purposes as well as collecting data from the handle about things like physical environment, use metrics, etc.

Once all hardware has been affixed to the door and power has been applied, the handle assembly 20 will initiate for three seconds allowing the sensors to calibrate and allow the system to become active. Once the initialization is complete the handle assembly 20 will switch to a predetermined color on the visual spectrum to signify the handle is clean and no further action is needed. At that time, the UV light is inactive and the handle assembly 20 is in standby mode waiting for a trigger event or a scheduled cleaning. The handle assembly 20 is now in state one. A trigger event is defined by the handle assembly 20 detecting proximity or contact between an object and the handle assembly 20. Once this takes place, the handle assembly 20 will switch to a predetermined color on the visual spectrum to signify that something has caused a trigger event. This time is defined as state two. During this time, the UV light is deactivated allowing for the contact to be broken and the user to have moved away from the handle assembly 20. Once the time delay has been satisfied and if there is no presence detected of the object within the proximity or the door is not moving, the handle assembly 20 will move into a self-cleaning cycle (between 30-120 sec in length). If the object is still detected within the proximity, the delay restarts and waits for the object to clear the proximity. Likewise, if the door is opened, the handle assembly 20 waits for the door to move back to steady state and will reinitiate the cleaning delay. This loop continues until the object has cleared the proximity or the door has stopped moving. The cleaning cycle is defined as state three. Once the cleaning cycle has initiated, the UV light becomes active and the visual spectrum light is changed again to signify cleaning. If an object comes into the proximity of the handle assembly 20 during the cleaning cycle, the UV light is deactivated and the present handle assembly 20 returns to state two. Once the criteria above are met for cleaning, the handle assembly 20 will move back to state three and start cleaning again. Once the cleaning cycle is finished, the handle assembly 20 returns to state one and awaits the next user. UV light has been widely proven to deactivate harmful pathogens. One of the main technical issues with effectively using UV light is the energy decay over distance. By imbedding the light source within the handle assembly 20, the decay rate is mitigated allowing the handle assembly 20 to clean at exceptional rates and use the maximum amount of energy produced for cleaning. Another unique feature is the safety aspect. The idea that UV light is minimally damaging to human tissue is mainstream. To error on the safe side, using sensing technology to mitigate and remove the ability of human harm is a pillar of this design.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional implementations that also incorporate the recited features. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

Also for purposes of this disclosure, the terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, the terms “upper,” “lower,” “right,” “left,” “rear,” “front,” “vertical,” “horizontal,” “inboard,” “outboard” and derivatives thereof shall relate to the orientation shown in FIG. 1. However, it is to be understood that various alternative orientations may be provided, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Changes and modifications in the specifically described embodiments may be carried out without departing from the principles of the present invention, which is intended to be limited only by the scope of the appended claims as interpreted according to the principles of patent law. The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. A handle assembly comprising:
a first support portion;
a second support portion;
a rod portion extending between the first support portion and the second support portion and having an outer surface,
a sensor array disposed in one of the first or second support portions and configured to detect if the handle assembly has been used; and
a light board configured to produce germicidal UV light upon detection from the sensor array that the handle assembly has been used such that the handle assembly is configured to automatically sanitize the outer surface of the rod portion after the handle assembly has been used.

2. The handle assembly of claim 1, wherein the outer surface of the rod portion is comprised of quartz.

3. The handle assembly of claim 2, wherein the outer surface of the rod portion is a rough surface.

4. The handle assembly of claim 1, wherein germicidal UV light is projected from the light board onto the rod portion in a direction perpendicular to the outer surface.

5. The handle assembly of claim 4, wherein the germicidal UV light is projected using fiber optics to transmit light to the outer surface of the rod portion.

6. The handle assembly of claim 5, wherein the germicidal UV light projected on to the outer surface of the rod portion comprises UV-C and visible light into the rod portion.

7. The handle assembly of claim 1, wherein the sensor array comprises a time of flight sensor or a radar sensor to sense distance.

8. The handle assembly of claim 1, wherein the first portion further comprises a light board including UV germicidal LEDs, light Spectrum LEDs, and a light sensor configured to sense ambient and UV light.

9. The handle assembly of claim 1, wherein the first portion further comprises an electronics carrier.

10. The handle assembly of claim 1, wherein the first portion further comprises an electronics cover configured to cover the electronics carrier.

11. The handle assembly of claim 1, where in the first portion further comprises an electronics board configured to control operation of the handle assembly.

12. The handle assembly of claim 11, wherein the electronics board further comprises control electronics, voltage regulators, and a motion sensor.

13. The handle assembly of claim 1, wherein the first portion further comprises a stanchion.

14. The handle assembly of claim 1, wherein the first portion further comprises a sensor cover comprised of a material capable of transmitting IR light and electronic signals for proximity sensing.

15. The handle assembly of claim 1, wherein the rod is comprised of fused silica.
